(19) United States
(12) Patent Application Publication
(10) Pub. No.: US 2021/0077570 A1
(43) Pub. Date: Mar. 18, 2021

(54) COMPOSITION COMPRISING A POLYMYXIN AND TEICOPLANIN

(71) Applicant: GALENUS G.H. AG, Hergiswil (CH)

(72) Inventors: Rita DOBMEYER, Hergiswil (CH); Kurt NABER, Straubing (DE)

(21) Appl. No.: 16/971,867

(22) PCT Filed: Feb. 17, 2019
(86) PCT No.: PCT/EP2019/053908

§ 371 Date: Aug. 21, 2020

(30) Foreign Application Priority Data
Feb. 23, 2018 (EP) 18158482.2
Jun. 15, 2018 (EP) 18178145.1

(51) Int. Cl. A61K 38/12; A61K 38/14; A61P 31/04

(52) U.S. Cl. CPC A61K 38/12; A61P 31/04; A61K 38/14

(57) ABSTRACT

The invention relates to a pharmaceutical composition comprising a polymyxin or a pharmaceutically acceptable salt or prodrug thereof, teicoplanin or a pharmaceutically acceptable salt thereof and a pharmaceutically acceptable excipient and to ready-to-use kits for the preparation of said pharmaceutical composition.

### CROSS-REFERENCE TO RELATED APPLICATIONS

This application claims the benefit from the priorities of European patent applications EP 18158482.2 of Feb. 23, 2018 and EP 18178145.1 of Jun. 15, 2018; the entire of these applications is hereby incorporated by reference.

### BACKGROUND OF THE INVENTION

### 1. Technical Field

The present invention relates to a pharmaceutical composition comprising a polymyxin or a pharmaceutically acceptable salt or prodrug thereof, teicoplanin and a pharmaceutically acceptable excipient and to a ready-to-use kit of parts for the preparation of such a pharmaceutical composition.

### 2. Background Information

Before the introduction of antibiotics, patients suffering from acute microbial infections (e.g. tuberculosis or pneumonia) had a low chance of survival. For example, mortality from tuberculosis was around 50%. Although the introduction of antimicrobial agents in the 1940s and 1950s rapidly changed this picture, bacteria have responded by progressively gaining resistance to commonly used antibiotics. Now, every country in the world has antibiotic-resistant bacteria. Indeed, more than 70% of bacteria that give rise to hospital acquired infections in the USA resist at least one of the main antimicrobial agents that are typically used to fight infection (Coates et al 2002 [1]).

One way of tackling the growing problem of resistant bacteria is the development of new classes of antimicrobial agents. However, until the introduction of linezolid in 2000, there had been no new class of antibiotic marketed for over 37 years. Moreover, even the development of new classes of antibiotic provides only a temporary solution, and indeed there are already reports of resistance of certain bacteria to linezolid (Gonzales et al 2001 [2] and Tsiodras et al 2001 [3]).

In order to develop more long-term solutions to the problem of bacterial resistance, it is clear that alternative approaches are required. One such alternative approach is to minimize, as much as is possible, the opportunities that bacteria are given for developing resistance to important antibiotics. Thus, strategies that can be adopted include limiting the use of antibiotics for the treatment of non-acute infections, as well as controlling which antibiotics are fed to animals in order to promote growth. However, in order to tackle the problem more effectively, it is necessary to gain an understanding of the actual mechanisms by which bacteria generate resistance to antibiotic agents. To do this requires first a consideration of how current antibiotic agents work to kill bacteria. Antimicrobial agents target essential components of bacterial metabolism. For example, the β-lactams (e.g. penicillins and cephalosporins) inhibit cell wall synthesis, whereas other agents inhibit a diverse range of targets, such as DNA gyrase (quinolones) and protein synthesis (e.g. macrolides, aminoglycosides, tetracyclines and oxazolidinones). The range of organisms against which the antimicrobial agents are effective varies, depending upon which organisms are heavily reliant upon the metabolic step(s) that is/are inhibited. Further, the effect upon bacteria can vary from a mere inhibition of growth (i.e. a bacteriostatic effect, as seen with agents such as the tetracyclines) to full killing (i.e. a bactericidal effect, as seen, e.g. with penicillin).

The combination of colistin (polymyxin E) and teicoplanin has shown beneficial effect in a Galleria mellonella model (Hornsey et al 2011 [4]) and a murine sepsis model caused by a colistin-susceptible Acinetobacter baumanii strain (Cirione et al 2016 [5]). In vitro studies have shown that the combination of colistin and teicoplanin is also synergistically active against colistin-resistant A. baumanii strains (Claeys et al 2014 [6], Bae et al 2016 [7]). However, neither an antibacterial synergism of a combination of colistin and teicoplanin has been described on colistin-susceptible and -resistant Pseudomonas aeruginosa strains or other Gram-negative bacteria, which all are resistant to teicoplanin mono nor an antibacterial synergism of a combination of colistin and teicoplanin against any Gram-positive bacteria.

Furthermore, it has not been described that the combination of colistin and teicoplanin reduces the emergence of bacterial resistance in Gram-negative bacteria, such as further Acinetobacter spp., which are different from A. baumanii, and Stenotrophomonas maltophilia, and other Gram-negatives, as compared to monotherapy with colistin, nor that the combination of colistin and teicoplanin reduces the emergence of bacterial resistance in Gram-positive bacteria, such as Staphylococcus aureus, as compared to monotherapy with teicoplanin. In addition, a combination therapy of colistin and teicoplanin has the advantage of the well known additive antibacterial effect with colistin against infections caused by Gram-negative bacteria in general and with teicoplanin against infections caused by Gram-positive bacteria, such as MSSA, MRSA, streptococci and enterococci. Such a broad-spectrum therapy may be indicated for initial empiric therapy in patients with severe infections but with unknown causative pathogens, where Gram-negatives and Gram-positives may also be suspected.

Accordingly, a combination therapy with colistin and teicoplanin is suggested using recommended or lower dosages of colistin against infections caused or suspected by multidrug resistant (MDR) Gram-negative-bacteria, such as Acinetobacter calcoaceticus, Acinetobacter guillouiae, Acinetobacter haemolyticus, Acinetobacter johnsonii, Acinetobacter junii, Acinetobacter lwoffii, Acinetobacter nosocomialis, Acinetobacter pitii, Acinetobacter radioresistens, Acinetobacter tjernbergiae, Acinetobacter ursingii, and Stenotrophomonas maltophilia, and dosages of teicoplanin usually recommended for infections caused by Gram-positive bacteria. This combination therapy is then also suitable for initial empiric therapy in severe infections with unknown causative pathogens, where Gram-negative and Gram-positive bacteria may also be suspected or demonstrated.

According to European Medicines Agency (EMA) completed review of polymyxin-based medicines (EMA/643444/2014 [8]) in patients with normal renal function (creatinine clearance≥80 ml/min) EMA approved a daily dose of colistin of 9 Million International Units (MIU) (approximately 300 mg colistin base activity-CBA) divided in 2 (bid) or 3 (tid) doses, whereas the US Food and Drug Administration (FDA) approved daily dose is 2.5-5 mg CBA/kg in patients with normal renal function (Nation et al 2016 [9]).

In the light of the above, a new approach to combating the problem of bacterial resistance might be to select and develop combination therapy with well know older antimicrobial agents to treat microorganisms which might be resistant to each one in monotherapy but not when using combination therapy. The production of such combination therapy would also allow, amongst other things, for the shortening of chemotherapy regimes in the treatment of microbial infections, thus reducing the frequency with which genotypic resistance arises in microorganisms.

### BRIEF SUMMARY OF THE INVENTION

Accordingly, in one embodiment of the present invention there is provided the use of combination of a polymyxin or a pharmaceutically acceptable salt or prodrug thereof, teicoplanin or a pharmaceutically acceptable salt thereof for the treatment or prevention of microbial infections caused by one or more Gram-negative bacteria selected from the group consisting of Acinetobacter calcoaceticus, Acinetobacter guillouiae, Acinetobacter haemolyticus, Acinetobacter johnsonii, Acinetobacter junii, Acinetobacter lwoffii, Acinetobacter nosocomialis, Acinetobacter pitii, Acinetobacter radioresistens, Acinetobacter tjernbergiae, Acinetobacter ursingii, and Stenotrophomonas maltophilia.

In a further embodiment of the invention there is provided a pharmaceutical composition comprising a polymyxin or a pharmaceutically acceptable salt or prodrug thereof, teicoplanin or a pharmaceutically acceptable salt thereof and a pharmaceutically acceptable excipient for the treatment or prevention of microbial infections caused by one or more Gram-negative bacteria selected from the group consisting of Acinetobacter calcoaceticus, Acinetobacter guillouiae, Acinetobacter haemolyticus, Acinetobacter johnsonii, Acinetobacter junii, Acinetobacter lwoffii, Acinetobacter nosocomialis, Acinetobacter pitii, Acinetobacter radioresistens, Acinetobacter tjernbergiae, Acinetobacter ursingii, and Stenotrophomonas maltophilia.

In a further embodiment, the invention provides a method of treating a microbial infection, caused by Gram-negative bacteria selected from the group consisting of Acinetobacter calcoaceticus, Acinetobacter guillouiae, Acinetobacter haemolyticus, Acinetobacter johnsonii, Acinetobacter junii, Acinetobacter lwoffii, Acinetobacter nosocomialis, Acinetobacter pitii, Acinetobacter radioresistens, Acinetobacter tjernbergiae, Acinetobacter ursingii, and Stenotrophomonas maltophilia, which method comprises administering teicoplanin or an acceptable salt thereof and a polymyxin selected from polymyxin B and (polymyxin E (colistin), or an acceptable salt or prodrug thereof in combination to poultry, mammals, including human beings.

The present invention is also based upon the unexpected finding that the activity of the combinations described herein is substantially improved compared to when either are administered alone.

Moreover, the combinations have surprisingly been shown to exhibit synergistic antimicrobial activity against log phase (i.e. multiplying) and/or clinically latent microorganisms.

The surprising biological activity of the combinations of the present invention offers the opportunity to shorten chemotherapy regimens and/or lower the dosage and may also result in a reduction in the emergence of microbial resistance associated with the use of such combinations.

In a further embodiment, the invention provides a kit of parts for the preparation of a pharmaceutical composition according to this invention essentially consisting of

(A) a first compartment containing a pharmaceutical composition comprising a polymyxin selected from polymyxin B and polymyxin E (colistin), or a pharmaceutically acceptable salt or prodrug thereof and a pharmaceutically acceptable excipient;

(B) a second compartment containing a pharmaceutical composition comprising teicoplanin or a pharmaceutically acceptable salt thereof and a pharmaceutically acceptable excipient;

(C) optionally a leaflet describing the dosage and administration of each of the pharmaceutical compositions (A) and (B).

In an alternative embodiment, the invention provides a kit of parts for the preparation of a pharmaceutical composition according to this invention essentially consisting of

(I) a first compartment containing a polymyxin selected from polymyxin B and polymyxin E (colistin), or a pharmaceutically acceptable salt or prodrug thereof in admixture with teicoplanin or a pharmaceutically acceptable salt thereof and optionally a pharmaceutically acceptable carrier;

(II) a second compartment containing an aqueous diluent for preparation of an injectable solution of; the mixture of the said polymyxin and teicoplanin of (I);

(III) optionally a leaflet describing preparation, the dosage and administration of the resulting injectable solution obtained by the combination of the components of compartments (I) and (II).

### DETAILED DESCRIPTION OF THE INVENTION

As used herein, the term “prodrug” relates to compounds which are quickly transformed in vivo into pharmacologically active compounds. The design of prodrugs is generally studied in Hardma et al. (Eds.), Goodman and Gilman's The Pharmacological Basis of Therapeutics, 9th ed., pages 11-16 (1996). An in-depth study is carried out in Higuchi et al., Prodrugs as Novel Delivery Systems, Vol. 14, ASCD Symposium Series, and in Roche (ed.), Bioreversible Carriers in Drug Design, American Pharmaceutical Association and Pergamon Press (1987). In a preferred embodiment colistimethate sodium (CMS) is a prodrug of colistin.

As used herein, the term “pharmaceutically acceptable salts” includes the metal salts or the addition salts which can be used in dosage forms. For example, the pharmaceutically acceptable salts of the compounds provided herein can be acid addition salts, base addition salts or metal salts, and can be synthesized from parent compounds containing a basic or acid residue by means of conventional chemical processes. Such salts are generally prepared, for example, by reacting the free acid or base forms of these compounds with a stoichiometric amount of the suitable base or acid in water or in an organic solvent or in a mixture of both. Non-aqueous media are generally preferred, such as ether, ethyl acetate, ethanol, isopropanol, or acetonitrile. Examples of acid addition salts include mineral acid additions salts such as, for example, hydrochloride, hydrobromide, hydroiodide, sulfate, nitrate, phosphate, organic acid addition salts such as, for example, acetate, maleate, fumarate, citrate, oxalate, succinate, tartrate, malate, mandelate, methanesulfonate and p-toluenesulfonate. Examples of alkali addition salts include inorganic salts such as, for example, ammonium salts and organic alkaline salts such as, for example, diethylamine, ethylenediamine, ethanolamine, N,N-dialkylenethanolamine, triethanolamine, glutamine and basic amino acid salts. Examples of metal salts include, for example, sodium, potassium, calcium, magnesium, aluminium and lithium salts.

As used herein, the term “pharmaceutically acceptable” relates to molecular entities and compositions that are physiologically tolerable and do not normally cause an allergic reaction or a similar adverse reaction, such as gastric discomfort, dizziness and the like, when administered to humans. As used herein, the term “pharmaceutically acceptable” preferably means that it is approved by a regulatory agency of the federal or state government or listed in the US pharmacopoeia or another pharmacopoeia, generally recognized for its use in animals, preferably in mammals and more particularly in human beings.

As used herein, the term “in combination with” covers both separate and sequential administration of the anti-retroviral agent and antimicrobial agent. For example, when the agents are administered sequentially, either the teicoplanin or the polymyxin may be administered first. When administration is simultaneous, the agents may be administered either in the same or a different pharmaceutical composition. Adjunctive therapy, i.e. where one agent is used as a primary treatment and the other agent is used to assist that primary treatment, is also an embodiment of the present invention.

As used herein, the term “teicoplanin” refers to an antibiotic used in the prophylaxis and treatment of serious infections caused by Gram-positive bacteria, including methicillin-resistant staphylococci, e.g. Staphylococcus aureus, and vancomycin-resistant enterococci, e.g. Enterococcus faecalis and Enterococcus faecium. It is a semisynthetic glycopeptide antibiotic with a spectrum of activity similar to vancomycin. Its mechanism of action is to inhibit bacterial cell wall synthesis.

Teicoplanin is as a rule a mixture of five compounds of the following formula (I)

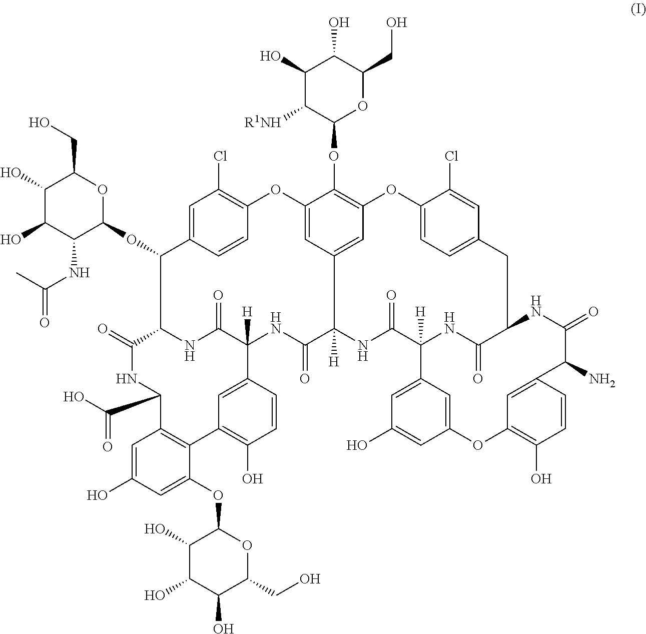

wherein

R¹ is a group selected from the formulae n-C₅H₁₁—CH═CH—(CH₂)₂—CO—, (CH₃)₂CH—(CH₂)₆—CO—, n-C₉H₁₉—CO—, C₂H₅—CH(CH₃)—(CH₂)₆—CO— and (CH₃)₂CH—(CH₂)₇—CO—

As used herein, the terms “polymyxin” or “polymyxins” relates to antibiotics, which are eventually neurotoxic and nephrotoxic, so are usually used only as a last resort if modern antibiotics are ineffective or are contraindicated. Typical uses are for infections caused by strains of multiple drug-resistant Pseudomonas aeruginosa, Acinetobacter spp. or carbapenemase-producing Enterobacteriaceae. Polymyxins have less effect on Gram-positive organisms. Preferred polymyxins are polymyxin B and E (colistin). Polymyxin B is composed of polymyxins B1, B1-I, B2, B3, and B6. Polymyxins B1 and B2 are considered major components of formula (II)

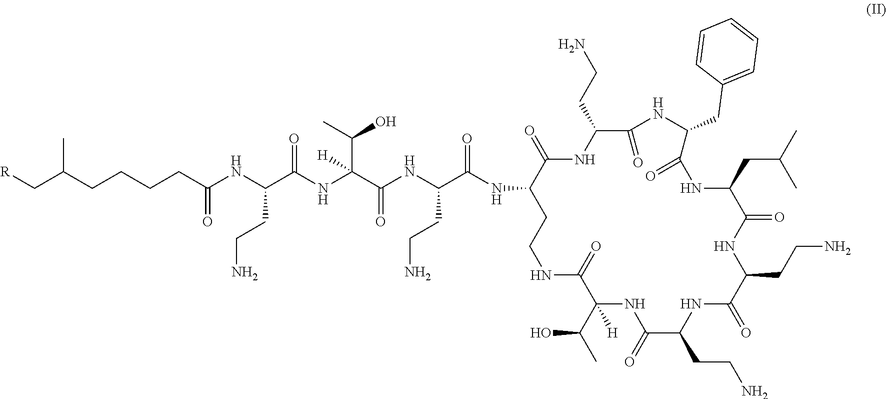

wherein R represents hydrogen (polymyxin B1) or methyl (polymyxin B2).

Polymyxin E (colistin) is a compound of formula (III)

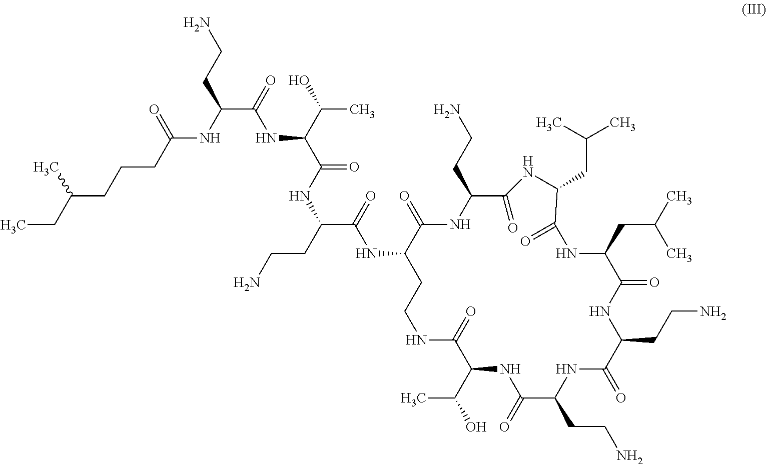 [SMILES: CCC(C)CCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](CC(C)C)NC(=O)[C@H](CCN)NC1=O)[C@@H](C)O]

Two forms of colistin are available commercially: colistin sulfate and colistimethate sodium (colistin methanesulfonate sodium, colistin sulfomethate sodium). Colistin sulfate is cationic; colistimethate sodium is anionic. Colistin sulfate is stable, but colistimethate sodium is readily hydrolysed to a variety of methanesulfonated derivatives.

According to a further embodiment of the invention, there is provided a product comprising teicoplanin and a polymyxin selected from polymyxin B and polymyxin E (colistin), as a combined preparation for simultaneous, separate or sequential use in treating microbial infections particularly by killing also microorganisms associated with a microbial infection, which are resistant to one component as monotherapy.

There is also provided a pharmaceutical composition comprising teicoplanin and a polymyxin selected from polymyxin B and polymyxin E (colistin), and a pharmaceutically acceptable adjuvant, diluent or carrier. Such a composition may be used for the treatment of microbial infections, in particular for killing also microorganisms associated with such infections, which are resistant to one component as monotherapy.

The combinations of the present invention are useful to treat microbial infections. In particular they may be used to kill also polymyxin-resistant microorganisms associated with microbial infections. References herein to the treatment of a microbial infection therefore include killing polymyxin-resistant microorganisms associated with such infections. Preferably, the combinations of the present invention are used to kill also polymyxin-resistant microorganisms associated with microbial infections.

As used herein, “kill” means a loss of viability as assessed by a lack of metabolic activity. As used herein, “clinically latent microorganism” means a microorganism that is metabolically active but has a growth rate that is below the threshold of infectious disease expression. The threshold of infectious disease expression refers to the growth rate threshold below which symptoms of infectious disease in a host are absent. The metabolic activity of clinically latent microorganisms can be determined by several methods known to those skilled in the art; for example, by measuring mRNA levels in the microorganisms or by determining their rate of uridine uptake. In this respect, clinically latent microorganisms, when compared to microorganisms under logarithmic growth conditions (in vitro or in vivo), possess reduced but still significant levels of:
(I) mRNA (e.g. from 0.0001 to 50%, such as from 1 to 30, 5 to 25 or 10 to 20%, of the level of mRNA); and/or
(II) uridine (e.g. [³H]uridine) uptake (e.g. from 0.0005 to 50%, such as from 1 to 40, 15 to 35 or 20 to 30% of the level of [³H]uridine uptake).

Clinically latent microorganisms typically possess a number of identifiable characteristics. For example, they may be viable but non-culturable; i.e. they cannot typically be detected by standard culture techniques, but are detectable and quantifiable by techniques such as broth dilution counting, microscopy, or molecular techniques such as polymerase chain reaction. In addition, clinically latent microorganisms are phenotypically tolerant, and as such are sensitive (in log phase) to the biostatic effects of conventional antimicrobial agents (i.e. microorganisms for which the minimum inhibitory concentration (MIC) of a conventional antimicrobial is substantially unchanged); but possess drastically decreased susceptibility to drug-induced killing.

In addition to the treatment of diseases caused by one or more Gram-negative bacteria selected from the group consisting of Acinetobacter calcoaceticus, Acinetobacter guillouiae, Acinetobacter haemolyticus, Acinetobacter johnsonii, Acinetobacter junii, Acinetobacter lwoffii, Acinetobacter nosocomialis, Acinetobacter pitii, Acinetobacter radioresistens, Acinetobacter tjernbergiae, Acinetobacter ursingii, and Stenotrophomonas maltophilia. the pharmaceutical composition according to the invention will also kill other microorganisms, which have infected the patient simultaneously.

As used herein, the term “microorganisms” means fungi and bacteria. References herein to “microbial”, “antimicrobial” and “antimicrobially” shall be interpreted accordingly. For example, the term “microbial” means fungal or bacterial, and “microbial infection” means any fungal or bacterial infection. Preferably, the term “microbial” in these contexts, means “bacterial.” As used herein, the term “bacteria” (and derivatives thereof, such as “microbial infection”) includes, but is not limited to, references to organisms (or infections due to organisms) of the following classes and specific types:

Gram-positive cocci, such as Staphylococci (e.g. Staph, aureus, Staph, epidermidis, Staph. saprophyticus, Staph. auricularis, Staph. capitis capitis, Staph. c. ureolyticus, Staph. caprae, Staph. cohnii cohnii, Staph. c. urealyticus, Staph. equorum, Staph. gallinarum, Staph. haemolyticus, Staph. hominis hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus), in particular methicillin-resistant Staphylococci; Streptococci (e.g. beta-haemolytic, pyogenic streptococci, such as Strept. agalactiae, Strept. canis, Strept. dysgalactiae dysgalactiae, Strept. dysgalactiae equisimilis, Strept. equi equi, Strept. equi zooepidemicus, Strept. iniae, Strept. porcinus and Strept. pyogenes), microaerophilic, pyogenic streptococci (Streptococcus “milleri”, such as Strept. anginosus, Strept. constellatus constellatus, Strept. constellatus pharyngidis and Strept. intermedius), oral streptococci of the “mitis” (alpha-haemolytic—Streptococcus “viridans”, such as Strept. mitis, Strept. oralis, Strept. sanguinis, Strept. cristatus, Strept. gordonii and Strept. parasanguinis), “salivarius” (non-haemolytic, such as Strept. salivarius and Strept. vestibularis) and “mutans” (tooth-surface streptococci, such as Strept. criceti, Strept. mutans, Strept. ratti and Strept. sobrinus) groups, Strept. acidominimus, Strept. bovis, Strept. faecalis, Strept. equinus, Strept. pneumoniae and Strept. suis, or Streptococci alternatively classified as Group A, B, C, D, E, G, L, P, U or V Streptococcus);

Gram-negative cocci, such as Neisseria gonorrhoeae, Neisseria meningitidis, Neisseria cinerea, Neisseria elongata, Neisseria flavescens, Neisseria lactamica, Neisseria mucosa, Neisseria sicca, Neisseria subflava and Neisseria weaveri;

Bacillaceae, such as Bacillus anthracis, Bacillus subtilis, Bacillus thuringiensis, Bacillus stearothermophilus and Bacillus cereus;

Enterobacteriaceae, such as Escherichia coli, Enterobacter (e.g. Enterobacter aerogenes, Enterobacter agglomerans and Enterobacter cloacae), Citrobacter (such as Citrob. freundii and Citrob. divernis), Hafnia (e.g. Hafnia alvei), Erwinia (e.g. Erwinia persicinus), Morganella morganii, Salmonella (Salmonella enterica and Salmonella typhi), Shigella (e.g. Shigella dysenteriae, Shigella flexneri, Shigella boydii and Shigella sonnei), Klebsiella (e.g. Klebs. pneumoniae, Klebs. oxytoca, Klebs. ornitholytica, Klebs. planticola, Klebs. ozaenae, Klebs. terrigena, Klebs. granulomatis (Calymmatobacterium granulomatis) and Klebs. rhinoscleromatis); Proteus (e.g. Pr. mirabilis, Pr. rettgeri and Pr. vulgaris), Providencia (e.g. Providencia alcalifaciens, Providencia rettgeri and Providencia stuartii), Serratia (e.g. Serratia marcescens and Serratia liquifaciens), and Yersinia (e.g. Yersinia enterocolitica, Yersinia pestis and Yersinia pseudotuberculosis);

Enterococci (e.g. Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus and Enterococcus solitarius);

Helicobacter (e.g. Helicobacter pylori, Helicobacter cinaedi and Helicobacter fennelliae); Acinetobacter baumanii;

Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzeri);

Bacteroides fragilis;

Peptococcus (e.g. Peptococcus niger); Peptostreptococcus;

Clostridium (e.g. C. perfringens, C. difficile, C. botulinum, C. tetani, C. absonum, C. argentinense, C. baratii, C. bifermentans, C. beijerinckii, C. butyricum, C. cadaveris, C. carnis, C. celatum, C. clostridioforme, C. cochlearium, C. cocleatum, C. fallax, C. ghonii, C. glycolicum, C. haemolyticum, C. hastiforme, C. histolyticum, C. indolis, C. innocuum, C. irregulare, C. leptum, C. limosum, C. malenominatum, C. novyi, C. oroticum, C. paraputrificum, C. piliforme, C. putrefasciens, C. ramosum, C. septicum, C. sordelii, C. sphenoides, C. sporogenes, C. subterminale, C. symbiosum and C. tertium);

Mycoplasma (e.g. M. pneumoniae, M. hominis, M. genitalium and M. urealyticum);

Mycobacteria (e.g. Mycobacterium tuberculosis, Mycobacterium avium, Mycobacterium fortuitum, Mycobacterium marinum, Mycobacterium kansasii, Mycobacterium chelonae, Mycobacterium abscessus, Mycobacterium leprae, Mycobacterium smegmitis, Mycobacterium africanum, Mycobacterium alvei, Mycobacterium asiaticum, Mycobacterium aurum, Mycobacterium bohemicum, Mycobacterium bovis, Mycobacterium branderi, Mycobacterium brumae, Mycobacterium celaturn, Mycobacterium chubense, Mycobacterium confluentis, Mycobacterium conspicuum, Mycobacterium cookii, Mycobacterium flavescens, Mycobacterium gadium, Mycobacterium gastri, Mycobacterium genavense, Mycobacterium gordonae, Mycobacterium goodii, Mycobacterium haemophilum, Mycobacterium hassicum, Mycobacterium intracellular, Mycobacterium interjectum, Mycobacterium heidelberense, Mycobacterium lentiflavum, Mycobacterium malmoense, Mycobacterium microgenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium szulgai, Mycobacterium terrae, Mycobacterium thermoresistabile, Mycobacterium triplex, Mycobacterium triviale, Mycobacterium tusciae, Mycobacterium ulcerans, Mycobacterium vaccae, Mycobacterium wolinskyi and Mycobacterium xenopi);

Haemophilus (e.g. Haemophilus influenzae, Haemophilus ducreyi, Haemophilus aegyptius, Haemophilus parainfluenzae, Haemophilus haemolyticus and Haemophilus parahaemolyticus);

Actinobacillus (e.g. Actinobacillus actinomycetemcomitans, Actinobacillus equuli, Actinobacillus hominis, Actinobacillus lignieresii, Actinobacillus suis and Actinobacillus ureae);

Actinomyces (e.g. Actinomyces israelii);

Brucella (e.g. Brucella abortus, Brucella canis, Brucella melintensis and Brucella suis);

Campylobacter (e.g. Campylobacter jejuni, Campylobacter coli, Campylobacter lari and Campylobacter fetus);

Listeria monocytogenes;

Vibrio (e.g. Vibrio cholerae and Vibrio parahaemolyticus, Vibrio alginolyticus, Vibrio carchariae, Vibrio fluvialis, Vibrio furnissii, Vibrio hollisae, Vibrio metschnikovii, Vibrio mimicus and Vibrio vulnificus);

Erysipelothrix rhusopathiae;

Corynebacteriaceae (e.g. Corynebacterium diphtheriae, Corynebacterium jeikeum and Corynebacterium urealyticum);

Spirochaetaceae, such as Borrelia (e.g. Borrelia recurrentis, Borrelia burgdorferi, Borrelia afzelii, Borrelia andersonii, Borrelia bissettii, Borrelia garinii, Borrelia japonica, Borrelia lusitaniae, Borrelia tanukii, Borrelia turdi, Borrelia valaisiana, Borrelia caucasica, Borrelia crocidurae, Borrelia duttoni, Borrelia graingeri, Borrelia hermsii, Borrelia hispanica, Borrelia latyschewii, Borrelia mazzottii, Borrelia parkeri, Borrelia persica, Borrelia turicatae and Borrelia venezuelensis) and Treponema (Treponema pallidum ssp. pallidum, Treponema pallidum ssp. endemicum, Treponema pallidum ssp. pertenue and Treponema carateum);

Pasteurella (e.g. Pasteurella aerogenes, Pasteurella bettyae, Pasteurella canis, Pasteurella dagmatis, Pasteurella gallinarum, Pasteurella haemolytica, Pasteurella multocida multocida, Pasteurella multocida gallicida, Pasteurella multocida septica, Pasteurella pneumotropica and Pasteurella stomatis);

Bordetella (e.g. Bordetella bronchiseptica, Bordetella hinzii, Bordetella holmseii, Bordetella parapertussis, Bordetella pertussis and Bordetella trematum);

Nocardiaceae, such as Nocardia (e.g. Nocardia asteroides and Nocardia brasiliensis);

Rickettsia (e.g. Ricksettsii or Coxiella burnetii);

Legionella (e.g. Legionalla anisa, Legionalla birminghamensis, Legionalla bozemanii, Legionalla cincinnatiensis, Legionalla dumoffii, Legionalla feeleii, Legionalla gormanii, Legionalla hackeliae, Legionalla israelensis, Legionalla jordanis, Legionalla lansingensis, Legionalla longbeachae, Legionalla maceachernii, Legionalla micdadei, Legionalla oakridgensis, Legionalla pneumophila, Legionalla sainthelensi, Legionalla tucsonensis and Legionalla wadsworthii); Moraxella catarrhalis; Cyclospora cayetanensis; Entamoeba histolytica; Giardia lamblia; Trichomonas vaginalis; Toxoplasma gondii; Burkholderia spp. such as Burkholderia cepacea, Burkholderia cenocepacia, Burkholderia multivorans, Burkholderia vietnamiensis, Burkholderia mallei and Burkholderia pseudomallei;

Francisella tularensis;

Gardnerella (e.g. Gardneralla vaginalis and Gardneralla mobiluncus);

Streptobacillus moniliformis;

Flavobacteriaceae, such as Capnocytophaga (e.g. Capnocytophaga canimorsus, Capnocytophaga cynodegmi, Capnocytophaga gingivalis, Capnocytophaga granulosa, Capnocytophaga haemolytica, Capnocytophaga ochracea and Capnocytophaga sputigena); Bartonella (Bartonella bacilliformis, Bartonella clarridgeiae, Bartonella elizabethae, Bartonella henselae, Bartonella quintana and Bartonella vinsonii arupensis);

Leptospira (e.g. Leptospira biflexa, Leptospira borgpetersenii, Leptospira inadai, Leptospira interrogans, Leptospira kirschneri, Leptospira noguchii, Leptospira santarosai and Leptospira weilii); Spirillium (e.g. Spirillum minus);

Baceteroides (e.g. Bacteroides caccae, Bacteroides capillosus, Bacteroides coagulans, Bacteroides distasonis, Bacteroides eggerthii, Bacteroides forsythus, Bacteroides fragilis, Bacteroides merdae, Bacteroides ovatus, Bacteroides putredinis, Bacteroides pyogenes, Bacteroides splanchinicus, Bacteroides stercoris, Bacteroides tectus, Bacteroides thetaiotaomicron, Bacteroides uniformis, Bacteroides ureolyticus and Bacteroides vulgatus);

Prevotella (e.g. Prevotella bivia, Prevotella buccae, Prevotella corporis, Prevotella dentalis {Mitsuokella dentalis), Prevotella denticola, Prevotella disiens, Prevotella enoeca, Prevotella heparinolytica, Prevotella intermedia, Prevotella loeschii, Prevotella melaninogenica, Prevotella nigrescens, Prevotella oralis, Prevotella oris, Prevotella oulora, Prevotella tannerae, Prevotella venoralis and Prevotella zoogleoformans);

Porphyromonas (e.g. Porphyromonas asaccharolytica, Porphyromonas cangingivalis, Porphyromonas canoris, Porphyromonas cansulci, Porphyromonas catoniae, Porphyromonas circumdentaria, Porphyromonas crevioricanis, Porphyromonas endodontalis, Porphyromonas gingivalis, Porphyromonas gingivicanis, Porphyromonas levii and Porphyromonas macacae);

Fusobacterium (e.g. F. gonadiaformans, F. mortiferum, F. naviforme, F. necrogenes, F. necrophorum necrophorum, F. necrophorum fundiliforme, F. nucleatum nucleatum, F. nucleatum fusiforme, F. nucleatum polymorphum, F. nucleatum vincentii, F. periodonticum, F. russii, F. ulcerans and F. varium);

Chlamydia (e.g. Chlamydia trachomatis);

Cryptosporidium (e.g. C. parvum, C. hominis, C. canis, C. felis, C. meleagridis and C. muris);

Chlamydophila (e.g. Chlamydophila abortus (Chlamydia psittaci), Chlamydophila pneumoniae (Chlamydia pneumoniae) and Chlamydophila psittaci (Chlamydia psittaci);

Leuconostoc (e.g. Leuconostoc citreum, Leuconostoc cremoris, Leuconostoc dextranicum, Leuconostoc lactis, Leuconostoc mesenteroides and Leuconostoc pseudomesenteroides);

Gemella (e.g. Gemella bergeri, Gemella haemolysans, Gemella morbillorum and Gemella sanguinis); and

Ureaplasma (e.g. Ureaplasma parvum and Ureaplasma urealyticum).

Particular bacteria that may be treated using a combination of the invention include: Gram negative bacteria;

Enterobacteriaceae, such as Escherichia coli, Klebsiella (e.g. Klebs. pneumoniae and Klebs. oxytoca) and Proteus (e.g. Pr. mirabilis, Pr. rettgeri and Pr. vulgaris);

Haemophilis influenzae;

Mycobacteria, such as Mycobacterium tuberculosis;

Pseudomonas, such as Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzeri);

Preferably, the bacterial infections treated by the combinations described herein are Gram-negative infections. Preferably, the bacterium is selected from the group consisting of Acinetobacter calcoaceticus, Acinetobacter guillouiae, Acinetobacter haemolyticus, Acinetobacter johnsonii, Acinetobacter junii, Acinetobacter lwoffii, Acinetobacter nosocomialis, Acinetobacter pitii, Acinetobacter radioresistens, Acinetobacter tjernbergiae, Acinetobacter ursingii, and Stenotrophomonas maltophilia, most preferred selected from the group consisting of Acinetobacter guillouiae, Acinetobacter haemolyticus, Acinetobacter junii, and Stenotrophomonas maltophilia. The combination of the present invention is particularly beneficial in treating (multi)-drug-resistant ((M)DR) bacteria. In all embodiments it is preferable that the combination therapy is synergistic as compared to the administration of the combination components taken alone. It should be kept in mind that although a combination such as that claimed may initially be demonstrated to be functional in treating (M)DR strains, they can then be used in treating non-resistant strains.

The combinations of the present invention may be used to treat infections associated with any of the above-mentioned bacterial organisms, and in particular they may be used for killing multiplying and/or clinically latent microorganisms associated with such an infection. In one aspect the invention provides the use of teicoplanin in combination with polymyxin B or polymyxin E (colistin), preferably colistin, for treating microbial infections, particularly for killing clinically latent microorganisms associated with a microbial infection.

Particular conditions which may be treated using the combination of the present invention include tuberculosis (e.g. pulmonary tuberculosis, non-pulmonary tuberculosis (such as tuberculosis lymph glands, genito-urinary tuberculosis, tuberculosis of bone and joints, tuberculosis meningitis) and miliary tuberculosis), anthrax, abscesses, acne vulgaris, actinomycosis, asthma, bacilliary dysentry, bacterial conjunctivitis, bacterial keratitis, bacterial vaginosis, botulism, Buruli ulcer, bone and joint infections, bronchitis (acute or chronic), brucellosis, burn wounds, cat scratch fever, cellulitis, chancroid, cholangitis, cholecystitis, cutaneous diphtheria, cystitis, diffuse panbronchiolitis, diphtheria, dental caries, diseases of the upper respiratory tract, eczema, empymea, endocarditis, endometritis, enteric fever, enteritis, epididymitis, epiglottitis, erysipelis, erysipelas, erysipeloid, erythrasma, eye infections, furuncles, gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leprosy, leptospirosis, listeriosis, liver abscesses, Lyme disease, lymphogranuloma venerium, mastitis, mastoiditis, meningitis and infections of the nervous system, mycetoma, nocardiosis (e.g. Madura foot), non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, phlegmons, pinta, plague, pleural effusion, pneumonia, postoperative wound infections, postoperative gas gangrene, prostatitis, pseudo-membranous colitis, psittacosis, pulmonary emphysema, pyelonephritis, pyoderma (e.g. impetigo), Q fever, rat-bite fever, reticulosis, ricin poisoning, Ritter's disease, salmonellosis, salpingitis, septic arthritis, septic infections, septicameia, sinusitis, skin infections (e.g. skin granulomas, impetigo, folliculitis and furunculosis), syphilis, systemic infections, tonsillitis, toxic shock syndrome, trachoma, tularaemia, typhoid, typhus (e.g. epidemic typhus, murine typhus, scrub typhus and spotted fever), urethritis, wound infections, yaws, aspergillosis, candidiasis (e.g. oropharyngeal candidiasis, vaginal candidiasis or balanitis), cryptococcosis, favus, histoplasmosis, intertrigo, mucormycosis, tinea (e.g. tinea corporis, tinea capitis, tinea cruris, tinea pedis and tinea unguium), onychomycosis, pityriasis versicolor, ringworm and sporotrichosis; or infections with MSSA, MRSA, Staph. epidermidis, Strept. agalactiae, Strept. pyogenes, Escherichia coli, Klebs. pneumoniae, Klebs. oxytoca, Pr. mirabilis, Pr. rettgeri, Pr. vulgaris, Ps. Aeruginosa, Haemophilis influenzae, Enterococcus faecalis and Enterococcus faecium. It will be appreciated that references herein to “treatment” extend to prophylaxis as well as the treatment of established diseases or symptoms.

The active ingredients may be used either as separate formulations or as a single combined formulation. When combined in the same formulation it will be appreciated that the two compounds must be stable and compatible with each other and the other components of the formulation.

Formulations of the invention include those suitable for oral, parenteral (including subcutaneous e.g. by injection or by depot tablet, intradermal, intrathecal, intramuscular e.g. by depot and intravenous), rectal and topical (including dermal, buccal and sublingual) or in a form suitable for administration by inhalation or insufflation administration. The most suitable route of administration may depend upon the condition and disorder of the patient. Preferably, the compositions of the invention are formulated for parenteral, inhalative or topical administration.

The formulations may conveniently be presented in unit dosage form and may be prepared by any of the methods well known in the art of pharmacy e.g. as described in “Remington: The Science and Practice of Pharmacy”, Lippincott Williams and Wilkins, 21^{st} Edition, (2005). Suitable methods include the step of bringing into association to active ingredients with a carrier which constitutes one or more excipients. In general, formulations are prepared by uniformly and intimately bringing into association the active ingredients with liquid carriers or finely divided solid carriers or both and then, if necessary, shaping the product into the desired formulation. It will be appreciated that when the two active ingredients are administered independently, each may be administered by a different means.

Formulations suitable for oral administration may be presented as discrete units such as capsules, cachets or tablets (e.g. chewable tablets in particular for pediatric administration), each containing a predetermined amount of active ingredient; as powder or granules; as a solution or suspension in an aqueous liquid or non-aqueous liquid; or as an oil-in-water liquid emulsion or water-in-oil liquid emulsion. The active ingredients may also be presented a bolus, electuary or paste.

Alternatively, the active ingredients may be incorporated into oral liquid preparations such as aqueous or oily suspensions, solutions, emulsions, syrups or elixirs. Formulations containing the active ingredients may also be presented as a dry product for constitution with water or another suitable vehicle before use. Such liquid preparations may contain conventional additives such as suspending agents (e.g. sorbitol syrup, methyl cellulose, glucose/sugar syrup, gelatin, hydroxymethyl cellulose, carboxymethyl cellulose, aluminium stearate gel and/or hydrogenated edible fats), emulsifying agents (e.g. lecithin, sorbitan mono-oleate and/or acacia), non-aqueous vehicles (e.g. edible oils, such as almond oil, fractionated coconut oil, oily esters, propylene glycol and/or ethyl alcohol), and preservatives (e.g. methyl or propyl p-hydroxybenzoates and/or sorbic acid).

Topical compositions, which are useful for treating disorders of the skin or of membranes accessible by digitation (such as membrane of the mouth, vagina, cervix, anus and rectum), include creams, ointments, lotions, sprays, gels and sterile aqueous solutions or suspensions. As such, topical compositions include those in which the active ingredients are dissolved or dispersed in a dermatological vehicle known in the art (e.g. aqueous or non-aqueous gels, ointments, water-in-oil or oil-in-water emulsions). Constituents of such vehicles may comprise water, aqueous buffer solutions, non-aqueous solvents (such as ethanol, isopropanol, benzyl alcohol, 2-(2-ethoxyethoxy)ethanol, propylene glycol, propylene glycol monolaurate, glycofurol or glycerol), oils (e.g. a mineral oil such as a liquid paraffin, natural or synthetic triglycerides such as Miglyol™, or silicone oils such as dimethicone). Depending, inter alia, upon the nature of the formulation as well as its intended use and site of application, the dermatological vehicle employed may contain one or more components selected from the following list: a solubilising agent or solvent (e.g. a β-cyclodextrin, such as hydroxypropyl β-cyclodextrin, or an alcohol or polyol such as ethanol, propylene glycol or glycerol); a thickening agent (e.g. hydroxymethyl cellulose, hydroxypropyl cellulose, carboxymethyl cellulose or carbomer); a gelling agent (e.g. a polyoxyethylene-polyoxypropylene copolymer); a preservative (e.g. benzyl alcohol, benzalkonium chloride, chlorhexidine, chlorbutol, a benzoate, potassium sorbate or EDTA or salt thereof); and pH buffering agent(s) (e.g. a mixture of dihydrogen phosphate and hydrogen phosphate salts, or a mixture of citric acid and a hydrogen phosphate salt). Topical formulations may also be formulated as a transdermal patch.

The most suitable route of administration may depend upon the condition and disorder of the patient.

When formulated with excipients, the active ingredients may be present in a concentration from 0.1 to 99.5% (such as from 0.5 to 95%) by weight of the total mixture; conveniently from 30 to 95% for tablets and capsules and 0.01 to 50% (such as from 3 to 50%) for liquid preparations.

Compositions for use according to the invention may be presented in a pack or dispenser device which may contain one or more unit dosage forms containing the active ingredients. The pack may, e.g. comprise a glass vial, a metal or plastic foil, such as a blister pack. Where the compositions are intended for administration as two separate compositions these may be presented in the form of a twin pack or a kit.

Compositions for inhalation will be administered by an inhaler (or puffer), which is a medical device used for delivering medication into the body via the lungs. Preferred inhalers are pressurized metered-dose inhalers (MDI), which are made up of 3 standard components—a metal canister, plastic actuator, and a metering valve, dry powder inhalers, which release a metered or device-measured dose of powdered medication and mechanically pressurized inhalers such as the Soft Mist Inhaler Respimat®.

Pharmaceutical compositions may also be prescribed to the patient in kit or “patient packs” containing the whole course of treatment in a single package, usually a blister pack or a pack of glass vials. Patient packs have an advantage over traditional prescriptions, where a pharmacist divides a patients' supply of a pharmaceutical from a bulk supply, in that the patient or the treating health professional always has access to the package insert contained in the patient pack, normally missing in traditional prescriptions. The inclusion of the package insert has been shown to improve patient compliance with the physician's instructions. The administration of the combination of the invention by means of a single patient pack, or patient packs of each composition, including a package insert directing the patient to the correct use of the invention is a desirable feature of this invention.

According to a further embodiment of the present invention there is provided a kit comprising at least one active ingredient of the combination according to the invention and an information insert containing directions on the use of the combination of the invention.

Suitable dosages and formulations for the administration of colistin are described in the product label for Colomycin® which can be found at https://www.medicines.org.uk/emc/medicine/1590

Suitable dosages and formulations for the inhalative administration of colistin are described in the SPC for colistimethate sodium Colobreathe® which can be found at http://www.ema.europa.eu/docs/en_GB/document_library/EPAR-Product_Information/human/001225/WC500123690.pdf Suitable dosages and formulations for the administration of teicoplanin are described in the product label for Targocid® 400 mg powder for solution for injection/infusion or oral solution which can be found at https://www.medicinesorg.uk/emc/medicine/27321

The route of administration and dosage of polymyxin B and polymyxin E (colistin) is generally determined by the administering physician. Typically, polymyxin B and polymyxin E (colistin) is administered by topical, intramuscular, intravenous, intrathecal, inhalative or ophthalmic routes depending on the nature of the bacterial infection.

The administration of the combination of the invention by means of a single patient pack, or patient packs of each composition, including a package insert directing the patient to the correct use of the invention is a desirable feature of this invention. According to a further embodiment of the present invention there is provided a patient pack comprising at least one active ingredient of the combination according to the invention and an information insert containing directions on the use of the combination of the invention.

The amount of active ingredients required for use in treatment will vary with the nature of the condition being treated and the age and condition of the patient, and will ultimately be at the discretion of the attendant physician or veterinarian. In general however, doses of the combined active ingredients employed for adult human treatment will typically be in the range of 0.02 to 5000 mg per day, preferably 1 to 1500 mg per day. The desired dose may conveniently be presented in a single dose or as divided doses administered at appropriate intervals, e.g. as two, three, four or more sub-doses per day.

Because of its very long terminal half-life of teicoplanin with a mean (±SD) of 157 (±93) hours (h) (Outman et al 1990 [10]) or with a median (range) of 168 (111-278) h (Antony et al 1991 [11]) the first 3-6 doses of 400 mg will be administered every 12 h, followed by 400 mg every 24 h (Outman et al 1990 [10], Antony et al 1991 [11], Zhou et al 2018 [12]). Preferably these doses of teicoplanin will be provided in form of a powder of teicoplanin with a carrier, preferably a salt such as sodium chloride for solution for injection or infusion.

Accordingly, the treatment kit also includes one combination of colistin 4.5 MIU, e.g. given as colistimethate sodium (CMS), a ‘prodrug’ that is converted to colistin in the body, plus teicoplanin 400 mg to be administered intravenously (IV) the first 3-6 doses every 12 h (bid). Thereafter a combination of colistin 4.5 MIU plus teicoplanin 200 mg to be administered IV every 12 h (bid). By this way the same colistin dose with half of the teicoplanin dose is further administered during steady state bid. If a lower dosage of colistin is appropriate, e.g. 3 MIU bid for treatment of complicated UTI, then a kit will be provided with vials containing colistin 3 MIU, e.g. as CMS, and teicoplanin 400 mg and 200 mg, respectively.

In case of severely ill patients where a loading dose of colistin up to 9 MIU is recommended (EMA 2014 [8]) 2 vials containing each colistin 4.5 MIU plus teicoplanin 400 mg (total colistin 9 MIU plus teicoplanin 800 mg) can be administered, because teicoplanin 800 mg as first loading dose is recommended in severe infections. Since the kit also contains vials with two different amounts of colistin (4.5 MIU and 3 MIU) combined either with teicoplanin 400 mg and 200 mg a more individual loading dose for colistin and teicoplanin is possible.

Since both, colistin and teicoplanin, are mainly excreted through the kidneys, in case of renal insufficiency the dosage reduction following the first normal dose can be made according to the degree of the individual renal insufficiency proportionally at about the same manner as recommended for colistin (Nation et al 2016 [9]).

### Biological Tests

Test procedures that may be employed to determine the biological (e.g. bactericidal or antimicrobial) activity of the active ingredients include those known to persons skilled in the art for determining:

(a) bactericidal activity against clinically latent bacteria; and

(b) antimicrobial activity against log phase bacteria.

In relation to (a) above, methods for determining activity against clinically latent bacteria include a determination, under conditions known to those skilled in the art (such as those described by Coates et al., 2002 [1]) of Minimum Stationary-cidal Concentration (“MSC”) or Minimum Dormicidal Concentration (“MDC”) for a test compound.

A typical method to determine their ability to kill clinically latent microorganisms may include the following steps:

(1) growing a bacterial culture to stationery phase;

(2) treating the stationery phase culture with one or more antimicrobial agents at a concentration and or time sufficient to kill growing bacteria, thereby selecting a phenotypically resistant sub-population;

(3) incubating a sample of the phenotypically resistant subpopulation with one or more test compounds or agents; and

(4) assessing any antimicrobial effects against the phenotypically resistant subpopulation. According to this method, the phenotypically resistant sub-population may be seen as representative of clinically latent bacteria which remain metabolically active in vivo and which can result in relapse or onset of disease.

In relation to (b) above, methods for determining activity against log phase bacteria include a determination, under standard conditions (i.e. conditions known to those skilled in the art), of Minimum Inhibitory Concentration (“MIC”) or Minimum Bactericidal Concentration (“MBC”) for a test compound.

The invention now being generally described, will be more readily understood by reference to the following Examples, which are included merely for purposes of illustration of certain aspects and embodiments of the present invention, and are not intended to limit the invention.

### EXAMPLES

### Example 1

Checkerboard analysis to determine minimal inhibitory concentration (MIC)/fractional inhibitory concentration (FIC) and minimal bacterial concentration (MBC)/fractional bacterial concentration (FBC) values of colistin sulphate (CS) plus teicoplanin (TP) for 6 Gram-negative strains with a bacterial load 5(1−10)*10⁵ CFU/ml in a cation-adjusted Mueller-Hinton broth (CAMHB).
a. Antimicrobial stock solutions were prepared using sterile distilled water
b. CS was added along the ordinate; TP was added along the abscissa to 96 well, flat bottom, polystyrene panels with no treatment. Colistin alone was tested in triplicates.
c. Serial 2-fold dilutions starting with 640 μg/ml CS and 640 μg/ml TP were made; 15 μl volume of the serial dilutions were added for each agent to the appropriate wells; final volume at 30 μl
d. For each agent tested alone an additional 15 μl media was added to the well. 30 μl were added in control wells where none of the agent was tested. The negative control well contains 150 μl media without any additions
e. From an overnight bacterial culture in tryptic soy broth, 100 μl was added to 10 mL of fresh tryptic soy broth. This was then incubated at 37° C. for 1.5 hour at 180 rpm.
f. Bacteria in 120 μl CAMHB were added to each well (end concentration 5(1−10)*10⁵ CFU/ml)
g. Plates were incubated at 37° C. in an ambient air incubator for 24 h
h. Turbidity at OD₆₀₀ is determined with a plate photometer
i. By using a one-time inoculator 3 μl from each well of the 96-well plate were transferred to three blood agar plates. The plates were incubated over night at 37° C. in an ambient air incubator
j. MIC values are defined as lowest concentration which restricts bacterial growth to OD₆₀₀≤0.1
k. MBC values were determined by visual reading the lowest concentration which inhibits 99.9% of the growth of bacteria in the wells
l. Determination of the fractional inhibitory concentration (FIC) index and fractional bactericidal concentration (FBC) index and characterization of antimicrobial interactions was assessed
   FIC calculation was carried out as: (MIC of drug A, tested in combination)/(MIC of drug A, tested alone)+(MIC of drug B, tested in combination)/(MIC of drug B, tested alone). FBC calculation was carried out just as well
   Interactive categories were calculated from checkerboard analysis using MIC results from testing each agent alone and MIC results from the combination wells adjacent to the wells with growth in them

a. Synergy; FIC/FBC≤0.5

b. Indifference; FIC/FBC>0.5 to <4.0

c. Antagonism; FIC/FBC≥4.0

Organisms:

a. A. junii 1391597 (IHMA), colistin resistant

b. A. nosocomialis 1461911 (IHMA), colistin resistant

c. A. haemolyticus 1655843 (IHMA), colistin resistant

d. A. guillouiae 1285286 (IHMA), colistin resistant

e. Stenotrophomonas maltophilia 1221783 (IHMA), colistin susceptible

The FIC/FBC of the test strains for TEC and CS are shown in the following Tables I and II:

TABLE 1
FIC indices from checkerboard titration synergy testing in CAMHB with inoculum 5*10⁵ CFU/ml

| | MIC [mg/l] | | MIC results for combination wells (FD/CS) and FIC indices (ΣFIC) | | | | | | | | ΣFIC | | |
|---|---|---|---|---|---|---|---|---|---|---|---|---|---|
| | TP | CS | result | ΣFIC | result | ΣFIC | result | ΣFIC | result | ΣFIC | Min | mean | Max |
| A. junii | >64 | 2 | 4/1 | 0.531 | 16/0.5 | 0.375 | 64/0.25 | 0.625 | | | 0.375 | 0.510 | 0.625 |
| 1397597 | | | | | | | | | | | | | |
| A. nosocomialis | >64 | 4 | 8/2 | 0.563 | 32/1 | 0.5 | 32/0.5 | 0.375 | | | 0.375 | 0.479 | 0.563 |
| 1461911 | | | | | | | | | | | | | |
| A. haemolyticus | >64 | >16 | 16/16 | 0.625 | 16/8 | 0.375 | 32/4 | 0.375 | 64/2 | 0.563 | 0.375 | 0.485 | 0.563 |
| 1655843 | | | | | | | | | | | | | |
| A. guillouiae | >64 | 2 | 4/1 | 0.531 | 8/0.5 | 0.313 | 64/0.25 | 0.625 | | | 0.313 | 0.490 | 0.625 |
| 1285286 | | | | | | | | | | | | | |
| S. maltophilia | >64 | 0.25 | 0.25/0.25 | 1.002 | | | | | | | 1.002 | | |
| 1221783 | | | | | | | | | | | | | |
| S. maltophilia | >64 (>64) | 0.5 | 32/0.25 | 0.75 | (1/4) | (0.508) | (2/2) | (0.266) | (32/1) | (0.375) | 0.75 | 0.75 | 0.75 |
| 1237289* | | (8) | | | | | | | | | (0.266) | (0.383) | (0.508) |

*S. maltophilia 1237289 grew very slow, therefor MIC/FIC values after 48 h growth are added in brackets

TABLE 2
FBC indices from checkerboard titration synergy testing in CAMHB with inoculum 5*10⁵ CFU/ml

| | MBC [mg/l] | | MIC results for combination wells (FD/CS) and FBC indices (ΣFBC) | | | | | | | | ΣFBC | | |
|---|---|---|---|---|---|---|---|---|---|---|---|---|---|
| | TP | CS | result | ΣFBC | result | ΣFBC | result | ΣFBC | result | ΣFBC | Min | mean | Max |
| A. junii | >64 | 8 | 0.25/4 | 0.502 | 0.5/2 | 0.254 | 8/1 | 0.188 | 32/0.5 | 0.313 | 0.188 | 0.314 | 0.502 |
| A. nosocomialis | >64 | 8 | 8/4 | 0.563 | 32/2 | 0.5 | 32/1 | 0.375 | 64/0.5 | 0.563 | 0.375 | 0.500 | 0.563 |
| A. haemolyticus | >64 | >16 | 64/16 | 1 | 64/8 | 0.75 | 64/4 | 0.625 | | | 0.625 | 0.792 | 1 |
| A. guillouiae | >64 | 4 | 0.5/2 | 0.504 | 8/1 | 0.313 | 32/0.5 | 0.375 | | | 0.313 | 0.396 | 0.504 |
| S. maltophilia | >64 | 2 | 32/1 | 0.75 | 32/0.5 | 0.5 | 32/0.25 | 0.375 | | | 0.375 | 0.542 | 0.75 |
| S. maltophilia | >64 | 8 | 1/4 | 0.508 | 2/2 | 0.266 | 32/1 | 0.375 | | | 0.266 | 0.383 | 0.508 |

Conclusions

MIC values of colistin were ≥4 mg/l for Acinetobacter nosocomialis 1461911 and A. haemolyticus 1655843 classifying them as colistin resistant according to EUCAST breakpoints. MIC values for A. junii 1397597 and A. guillouiae 1655843 were 2 mg/l. Since EUCAST breakpoints are mg/l=sensitive and >2 mg/l, both strains are difficult to classify. After 24 h growth both S. maltophilia strains showed a MIC value of colistin 0.5 mg/l, classifying them as colistin sensitive. However, S. maltophilia 1237289 grew very slowly. After 48 h incubation, this strain had an MIC value of 8 mg/l. Checkerboard assays showed synergy between TP and CS for the A. junii 1397597, A. haemolyticus 1655843, A. guillouiae 1655843 based on both, inhibitory and bactericidal levels. For A. nosocomialis 1461911, S. maltophilia 1237289 and S. maltophilia 1221783 synergy could be shown only for one, either FIC or FBC values.

### Example 2

Elimination of Stationary Bacteria from Biofilm

To test the elimination of stationary bacteria from biofilm in vitro in CAMHB comparing combination versus monotherapy the same teicoplanin and colistin (serum) concentrations derived from pharmacokinetic studies are used as above.

Each of the total 16 sets as above is performed in duplicates. Each tube is filled with 5 ml CAMHB and in one of each duplicates a 2 cm long piece of a plastic catheter is inserted. Then all tubes are inoculated with the test strain and incubated aerobically at ambient air at 37° C. overnight (16-24 h). The tubes are removed from the incubator and the appropriate blank, mono or combination drug concentrations to be tested are added to each tube and the tubes are incubated aerobically at ambient air at 37° C. for an additional 24 h. The tubes are removed from the incubator and vortexed for about 10 min. Thereafter the catheter pieces from the corresponding one of the duplicates are removed.

From tubes showing growth with turbidity the MIC of CS or TEC in case of testing Gram-positive bacteria for the test strains is determined as above and compared to the original MIC. All tubes showing growth or no growth according to turbidity are centrifuged for 10 min at 3000 rpm and about 100 μL of the sediment is transferred onto IsoSensitest agar plate supplemented with 5% blood (Oxoid) and containing concentrations of CS of 4 mg/L, 8 mg/L, 16 mg/L, and 32 mg/L—or TEC of 4 mg/L, 8 mg/L, 16 mg/L in case a Gram-positive strain is tested. After overnight incubation the number of CFUs growing on the plates containing different CS concentrations—or TEC concentrations in case a Gram-positive strain is tested—are counted.

Results

The tubes showing no gross turbidity and containing any combination of TEC and CS do not show after centrifugation any differences in colony counts or increase in MIC of CS—or TEC in case a Gram-positive strain is tested—for any of the strains tested whether incubated with or without a catheter piece. In contrast the tubes showing no gross turbidity and containing only CS as mono substance—or TEC as mono substance in case a Gram-positive strain is tested—show higher colony counts for any of the strains tested and in about one third an increase (at least two dilution steps) of MIC of CS—or TEC in case a Gram-positive strain is tested—if incubated with as compared without a catheter piece.

Conclusions

The combination of TEC and CS does not only prevent emergence of resistance as compared to CS alone—or TEC alone in case a Gram-positive strain is tested—but is also able to eliminate bacteria from biofilm whereas this is not so well the case with CS alone—or TEC alone in case a Gram-positive strain is tested.

### Example 3

Elimination of Dormant (Resistant) Bacteria

In order to test the elimination of dormant (resistant) bacteria the long term non-multiplying stationary phase model is used (Hu et al 2010 [18]). Long-term stationary phase cultures of 5 to 6 days are chosen with the test strains as described above. These late stationary phase cultures, may represent a mixed population with a dynamic balance of cell division and cell death. In order to induce the bacterial cells into a non-replicating stage, the cells are washed and incubated with PBS. No changes in CFU counts of the bacteria are observed after 24 hours of incubation in the buffer (data not shown) indicating that the bacteria are in a non-multiplying state. The nutrient depleted 5-6 day stationary phase cell suspension now called non-multiplying stationary phase bacteria is used to test TEC and CS as mono substances or in combinations with the concentrations as mentioned above.

Results and Conclusions

The results show that only the drugs TEC and CS in combination, but not as mono substances are able to eliminate such non-multiplying stationary phase bacteria.

### REFERENCES

The following publications are hereby incorporated by reference in their entirety as if each individual publication is specifically and individually indicated to be incorporated by reference. In case of conflict, the present application, including any definitions herein, will control.
[1] Coates A, Hu Y, Bax R, Page C. The future challenges facing the development of new antimicrobial drugs. Nat Rev Drug Discov. 2002; 1(11):895-910.
[2] Gonzales R D, Schreckenberger P C, Graham M B, Kelkar S, DenBesten K, Quinn J P. Infections due to vancomycin-resistant Enterococcus faecium resistant to linezolid. Lancet. 2001 Apr. 14; 357(9263):1179.
[3] Tsiodras S, Gold H S, Sakoulas G, Eliopoulos G M, Wennersten C, Venkataraman L, Moellering R C, Ferraro M J. Linezolid resistance in a clinical isolate of Staphylococcus aureus. Lancet. 2001 Jul. 21; 358(9277):207-8.
[4] Hornsey M, Wareham D W. In vivo efficacy of glycopeptide-colistin combination therapies in a Galleria mellonella model of Acinetobacter baumannii infection. Antimicrob Agents Chemother. 2011 July; 55(7):3534-7. doi: 10.1128/AAC.00230-11. Epub 2011 Apr. 18.
[5] Cirioni O, Simonetti O, Pierpaoli E, Barucca A, Ghiselli R, Orlando F, Pelloni M, Trombettoni M M, Guerrieri M, Offidani A, Giacometti A, Provinciali M. Colistin enhances therapeutic efficacy of daptomycin or teicoplanin in a murine model of multiresistant Acinetobacter baumannii sepsis. Diagn Microbiol Infect Dis. 2016 December; 86(4):392-398. doi: 10.1016/j. diagmicrobio.2016.09.010. Epub 2016 Sep. 17.
[6] Claeys K C, Fiorvento A D, Rybak M J. A Review of Novel Combinations of Colistin and Lipopeptide or Glycopeptide Antibiotics for the Treatment of Multidrug-Resistant Acinetobacter baumannii. Infect Dis Ther. 2014 December; 3(2):69-81. doi: 10.1007/s40121-014-0051-9. Epub 2014 Dec. 5.
[7] Bae S, Kim M C, Park S J, Kim H S, Sung H, Kim M N, Kim S H, Lee S O, Choi S H, Woo J H, Kim Y S, Chong Y P. In Vitro Synergistic Activity of Antimicrobial Agents in Combination against Clinical Isolates of Colistin-Resistant Acinetobacter baumannii. Antimicrob Agents Chemother. 2016 Oct. 21; 60(11):6774-6779. doi: 10.1128/AAC.00839-16. Print 2016 November
[8] European Medicines Agency (EMA) completes review of polymyxin-based medicines. Recommendations issued for safe use in patients with serious infections resistant to standard antibiotics. EMA/643444/2014 (24 Oct. 2014). http://www.ema.europa.eu/ema/index.jsp?curl=pages/news_and_events/news/2014/10/news_d etail_002194.jsp&mid=WC0b01ac058004d5c1 (visited 27 Jan. 2018)
[9] Nation R L, Garonzik S M, Li J, Thamlikitkul V, Giamarellos-Bourboulis E J, Paterson D L, Turnidge J D, Forrest A, Silveira F P. Updated US and European Dose Recommendations for Intravenous Colistin: How Do They Perform? Clin Infect Dis. 2016 Mar. 1; 62(5):552-558. doi: 10.1093/cid/civ964. Epub 2015 Nov. 25.
[10] Outman W R, Nightingale C H, Sweeney K R, Quintiliani R. Teicoplanin pharmacokinetics in healthy volunteers after administration of intravenous loading and maintenance doses. Antimicrob Agents Chemother. 1990 November; 34(11):2114-7.
[11] Antony K K, Lewis E W, Kenny M T, Dulworth J K, Brackman M B, Kuzma R, Yuh L, Eller M G, Thompson G A. Pharmacokinetics and bioavailability of a new formulation of teicoplanin following intravenous and intramuscular administration to humans. J Pharm Sci. 1991 June; 80(6):605-7.
[12] Zhou L, Gao Y, Cao W, Liu J, Guan H, Zhang H, Shi Y, Lv W, Cheng L. Retrospective analysis of relationships among the dose regimen, trough concentration, efficacy, and safety of teicoplanin in Chinese patients with moderate-severe Gram-positive infections. Infection and Drug Resistance 2018:11 29-36
[13] CLSI. M07-A10. Methods for dilution antimicrobial susceptibility tests for bacteria that grow aerobically; approved standard—tenth edition. Wayne, Pa., Clinical and Laboratory Standards Institute, 2015.
[14] CLSI. M26-A. Methods for determining bactericidal activity of antimicrobial agents; approved guideline. Wayne, Pa., Clinical and Laboratory Standards Institute, 1999.
[15] ISO 20776-1. Clinical laboratory testing and in vitro diagnostic test systems—Susceptibility testing of infectious agents and evolution of performance of antimicrobial susceptibility test devices. Part 1: Reference method for testing the in vitro activity of antimicrobial agents against rapidly growing aerobic bacteria involved in infectious diseases. 2006
[16] Eliopoulos G M, and R. C. Moellering, Jr. 1996. Antimicrobial combinations, p. 330-396. In V. Lorian (ed.), Antibiotics in laboratory medicine, 4th ed. The Williams & Wilkins Co., Baltimore, Md.
[17] EUCAST—European Committee for Antimicrobial Susceptibility Testing of the European Society of Clinical Microbiology and Infectious Diseases (ESCMID). 2000. EUCAST Definitive Document E.Def 1.2, May 2000: Terminology relating to methods for the determination of susceptibility of bacteria to antimicrobial agents. Clin Microbiol Infect 6:503-8.
[18] Hu Y, Shamaei-Tousi A, Liu Y, Coates A. A new approach for the discovery of antibiotics by targeting non-multiplying bacteria: a novel topical antibiotic for staphylococcal infections. PLoS One. 2010; 5(7):e11818. doi: 10.1371/journal.pone.00118.

1. A pharmaceutical composition comprising a polymyxin or a pharmaceutically acceptable salt or prodrug thereof, teicoplanin or a pharmaceutically acceptable salt thereof and a pharmaceutically acceptable excipient for the treatment or prevention of microbial infections caused by one or more Gram-negative bacteria selected from the group consisting of Acinetobacter calcoaceticus, Acinetobacter guillouiae, Acinetobacter haemolyticus, Acinetobacter johnsonii, Acinetobacter junii, Acinetobacter lwoffii, Acinetobacter nosocomialis, Acinetobacter pitii, Acinetobacter radioresistens, Acinetobacter tjernbergiae, Acinetobacter ursingii, and Stenotrophomonas maltophilia.

2. A pharmaceutical composition according to claim 1, wherein the infections is caused by Gram-negative bacteria, which are colistin resistant.

3. A pharmaceutical composition according claim 1 or 2, wherein the polymyxin is selected from polymyxin B and colistin (polymyxin E).

4. A pharmaceutical composition according to one of claims 1 to 3, wherein teicoplanin is a mixture of five compounds of formula (I) 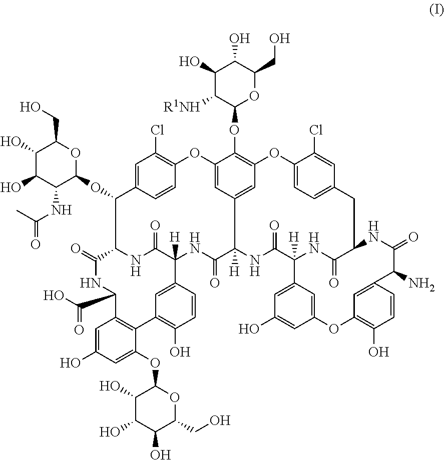
wherein
R¹ is a group selected from the formulae
n-C₅H₁₁—CH═CH—(CH₂)₂—CO—(CH₃)₂CH—(CH₂)₆—CO—,n-C₉H₁₉—CO—,C₂H₅—CH(CH₃)—(CH₂)₆—CO— and(CH₃)₂CH—(CH₂)₇—CO—

5. A pharmaceutical composition according to one of the claims 1 to 4, in which the concentration ratio of polymyxin to teicoplanin is from 10:1 to 1:10.

6. A pharmaceutical composition according to one of the claims 1 to 5 for the treatment of tuberculosis, anthrax, abscesses, acne vulgaris, actinomycosis, asthma, bacilliary dysentry, bacterial conjunctivitis, bacterial keratitis, bacterial vaginosis, botulism, Buruii ulcer, bone and joint infections, bronchitis (acute or chronic), brucellosis, burn wounds, cat scratch fever, cellulitis, chancroid, cholangitis, cholecystitis, cutaneous diphtheria, cystitis, diffuse panbronchiolitis, diphtheria, dental caries, diseases of the upper respiratory tract, eczema, empymea, endocarditis, endometritis, enteric fever, enteritis, epididymitis, epiglottitis, erysipelis, erysipelas, erysipeloid, erythrasma, eye infections, furuncles, gardnereila vaginitis, gastrointestinal infections (gastroenteritis), urogenital infections, urinary tract infections, gingivitis, gonorrhoea, granuloma inguinale, Haverhill fever, infected burns, infections following dental operations, infections in the oral region, infections associated with prostheses, intraabdominal abscesses, Legionnaire's disease, leprosy, leptospirosis, listeriosis, liver abscesses, Lyme disease, lymphogranuloma venerium, mastitis, mastoiditis, meningitis and infections of the nervous system, mycetoma, nocardiosis, non-specific urethritis, opthalmia, osteomyelitis, otitis, orchitis, pancreatitis, paronychia, pelveoperitonitis, peritonitis, peritonitis with appendicitis, pharyngitis, phlegmons, pinta, plague, pleural effusion, pneumonia, postoperative wound infections, postoperative gas gangrene, prostatitis, pseudo-membranous colitis, psittacosis, pulmonary emphysema, pyelonephritis, pyoderma, Q fever, rat-bite fever, reticulosis, ricin poisoning, Ritter's disease, salmonellosis, salpingitis, septic arthritis, septic infections, septicameia, sinusitis, skin infections, syphilis, systemic infections, tonsillitis, toxic shock syndrome, trachoma, tularaemia, typhoid, typhus, urethritis, wound infections, yaws, aspergillosis, candidiasis, cryptococcosis, favus, histoplasmosis, intertrigo, mucormycosis, tinea, onychomycosis, pityriasis versicolor, ringworm and sporotrichosis; or infections with Gram-positive bacteria, such as MSSA, MRSA, Staph. epidermidis, Strept. agalactiae, Strept. pyogenes, Enterococcus faecalis and Enterococcus faecium, or infections with Gram-negative bacteria, such as Escherichia coli, Klebsiella pneumoniae, Klebs. oxytoca, Proteus mirabiiis, Pr. rettgeri, Pr. vulgaris, Haemophilis influenzae.

7. A pharmaceutical composition according to one of claims 1 to 6, which comprises 1 Million International Units (MIU) to 9 MIU, corresponding to about 33 mg to 300 mg colistin base activity (CBA), of a polymyxin selected from polymyxin B and polymyxin E (colistin), or a pharmaceutically acceptable salt or prodrug thereof and 100 to 800 mg of teicoplanin.

8. A pharmaceutical composition according to one of claims 1 to 7, which comprises 3 MIU to 4.5 MIU, corresponding to about 100 to 150 mg CBA of a polymyxin selected from polymyxin B and (polymyxin E (colistin), or a pharmaceutically acceptable salt or prodrug thereof.

9. A pharmaceutical composition according to one of claims 1 to 8, which comprises 200 to 400 mg of teicoplanin or a pharmaceutically acceptable salt thereof.

10. A pharmaceutical composition according to one of the claims 1 to 9, which is in a form suitable for parenteral administration or for inhalation.

11. A pharmaceutical composition according to one of the claims 1 to 10, wherein the pharmaceutically acceptable excipient comprises one or more fluid or semi-solid vehicles selected from the group consisting of polymers, thickeners, buffers, neutralizers, chelating agents, preservatives, surfactants, emulsifiers, antioxidants, waxes, oils, emollients, solvents and penetration enhancers.

12. A method of killing multiplying bacteria associated with a Gram-negative bacterial infection which method comprises administering to a mammal having said Gram-negative bacterial infection a combination of a polymyxin or a pharmaceutically acceptable salt or prodrug thereof and teicoplanin or a pharmaceutically acceptable salt thereof, wherein the combination exhibits synergistic antibacterial activity against the multiplying bacteria caused by one or more Gram-negative bacteria selected from the group consisting of Acinetobacter calcoaceticus, Acinetobacter guillouiae, Acinetobacter haemolyticus, Acinetobacter johnsonii, Acinetobacter junii, Acinetobacter lwoffii, Acinetobacter nosocomialis, Acinetobacter pitii, Acinetobacter radioresistens, Acinetobacter tjernbergiae, Acinetobacter ursingii, and Stenotrophomonas maltophilia.

13. Teicoplanin for the use as a medicament which inhibits the resistance of Gram-negative bacteria against polymyxin antibiotics in combination with polymyxin B or polymyxin E (colistin) or a pharmaceutically acceptable salt or prodrug thereof for the

14. A ready-to-use kit of parts for the preparation of a pharmaceutical composition in accordance with the claims 1 to 11 essentially consisting of
(A) a first compartment containing a pharmaceutical composition comprising a polymyxin selected from polymyxin B and polymyxin E (colistin), or a pharmaceutically acceptable salt or prodrug thereof and a pharmaceutically acceptable excipient;
(B) a second compartment containing a pharmaceutical composition comprising teicoplanin, or a pharmaceutically acceptable salt thereof and a pharmaceutically acceptable excipient;
(C) optionally a leaflet describing the dosage and administration of each of the pharmaceutical compositions (A) and (B).

15. A kit of parts according to claim 14 comprising
(A) a first compartment containing a pharmaceutical composition comprising 1 MIU to 9 MIU, of a polymyxin selected from polymyxin B and polymyxin E (colistin), or a pharmaceutically acceptable salt or prodrug thereof and a pharmaceutically acceptable excipient; and
(B) a second compartment containing a pharmaceutical composition comprising 100 to 800 mg of teicoplanin, or a pharmaceutically acceptable salt thereof and a pharmaceutically acceptable excipient.

16. A kit of parts according to claim 14 or 15 wherein the components (A) and/or (B) consist of the sub-compartments
(A1) containing a polymyxin selected from polymyxin B and polymyxin E (colistin), or a pharmaceutically acceptable salt or prodrug thereof and optionally a solid carrier;
(A2) containing an aqueous diluent for preparation of an injectable solution of said polymyxin; and/or
(B1) containing teicoplanin, or a pharmaceutically acceptable salt thereof and optionally a solid carrier;
(B2) containing an aqueous diluent for preparation of an injectable solution of teicoplanin.
